(19) United States
(12) Patent Application Publication
(10) Pub. No.: US 2025/0161243 A1
(43) Pub. Date: May 22, 2025

(54) MEF2 TRANSCRIPTIONAL ACTIVATORS TO TREAT NEUROLOGIC CONDITIONS

(71) Applicant: The Scripps Research Institute, La Jolla, CA (US)

(72) Inventor: Stuart Lipton, Rancho Santa Fe, CA (US)

(21) Appl. No.: 18/839,561

(22) PCT Filed: Feb. 21, 2023
(86) PCT No.: PCT/US2023/062912

§ 371 Date: Aug. 19, 2024

Related U.S. Application Data
(60) Provisional application No. 63/268,301, filed on Feb. 21, 2022.

(51) Int. Cl. A61K 31/167

(52) U.S. Cl. CPC A61K 31/167

(57) ABSTRACT

Disclosed are compounds, and corresponding methods of treatment, that activate myocyte-specific enhancer factor 2 (MEF2) transcriptional activity that are therefore useful in treating deficits in MEF2C activity found in autism spectrum disorder (ASD), intellectual disability (ID), attention deficit and hyperactivity disorder (ADHD), and in diseases characterized by cognitive decline such as Alzheimer's disease (AD), Lewy body dementia (LED), Frontotemporal dementia (FTD), and other forms of dementia, as well as movement disorders such as Parkinson's disease (PD) and parkinsonism from other causes.

### CROSS REFERENCE TO RELATED APPLICATION

This application claims priority to U.S. provisional patent application No. 63/268,301, which was filed on Feb. 21, 2022, and which is hereby incorporated by reference in its entirety.

### FIELD OF THE INVENTION

The application is related to compounds, and corresponding methods of treatment, that activate myocyte-specific enhancer factor 2 (MEF2) transcriptional activity that are therefore useful in treating deficits in MEF2C activity found in autism spectrum disorder (ASD), intellectual disability (ID), attention deficit and hyperactivity disorder (ADHD), and in diseases characterized by cognitive decline such as Alzheimer's disease (AD), Lewy body dementia (LBD), Frontotemporal dementia (FTD), and other forms of dementia, as well as movement disorders such as Parkinson's disease (PD) and parkinsonism from other causes.

### BACKGROUND

The Myocyte enhancer factor 2 (MEF2) family of transcription factors regulate extensive programs of gene expression that factor prominently in the development and maintenance of many tissues, including the brain (A. Assali et al., Curr. Opin. Neurobiol. 59 (2019) 49-58). MEF2 proteins are regulated by neuronal synaptic activity, and they recruit several epigenetic enzymes to influence chromatin structure and gene expression during development and throughout adulthood (id.). The MEF2C isoform in particular regulates the expression, directly and indirectly, of more than a thousand genes in the developing brain (id.).

MEF2 transcription factors have also been associated with synaptic plasticity, the developmental mechanisms governing memory and learning, and with several neurologic conditions, like autism spectrum disorder (ASD). For instance, recent genomic findings have posited a link between MEF2 defects, particularly in the MEF2C isoform and ASD and intellectual disability (ID) (R. Chaudhary et al., Heliyon 7 (4) (2021) e06854). Further, in MEF2C-deficient mice, which constitute a useful model for human MEF2C haploinsufficiency syndrome (MCHS), the pharmacological intervention by an N-methyl-D-aspartate-type glutamate receptor (NMDAR) antagonist was shown to improve autistic/MCHS-like behavioral deficits (S. Tu et al., Nat. Commun. 8 (2017) 1488).

MEF2 transcription factors also play a role in the molecular mechanism underlying learning and memory and other processes involved in dementia and neurodegenerative disease, such as Alzheimer's disease and Parkinson's disease/Lewy body dementia, and Fragile X syndrome (J. Wang, Exploratory Research and Hypothesis in Medicine 1 (2016) 2-6); S. D. Ryan, et al., Cell 155 (2013) 1351-1364; S.-i. Okamoto, et al., Cell Rep 8 (2014) 217-228; S. J. Barker, et al., Sci Transl Med. 13 (2021) eabd7695). Fragile X syndrome (FXS) is a neurodevelopmental disorder characterized by intellectual disability (ID), ADHD, anxiety symptoms, epilepsy, and autism-related symptoms (T. M. Kazdoba, et al., Intractable Rare Dis. Res. 3 (4) (2014) 118-133). Neurodegenerative diseases are associated with cognitive impairments. However, few patients are resilient to such diseases and are capable of preserving normal cognition. The MEF2 transcriptional network also is over-represented among genes that are most predictive of end-stage cognition in individuals with Alzheimer's disease. Specifically, it has been shown that MEF2 expression is increased in cognitive-resilient patients, reinforcing the view that activation of MEF2 is a neuroprotective pathway for ameliorating ASD and cognitive impairments in neurodegenerative diseases such as Alzheimer's disease (S. J. Barker (2021)).

Recently, MEF2C dysfunction has also been implicated in the etiology of attention deficit and hyperactivity disorder (ADHD) (See: Fu, X., Yao, T., Chen, X., Li, H., & Wu, J. (2022). MEF2C gene variations are associated with ADHD in the Chinese Han population: a case-control study. J. Neural Transmission (Vienna, Austria: 1996), 129 (4), 431-439). Therefore, MEF activation may be a novel treatment for ADHD, representing another important unmet need in the field.

### REFERENCES

Demontis, D., Walters, G. B., Athanasiadis, G., Walters, R., Therrien, K., Nielsen, T. T., Farajzadeh, L., Voloudakis, G., Bendl, J., Zeng, B., Zhang, W., Grove, J., Als, T. D., Duan, J., Satterstrom, F. K., Bybjerg-Grauholm, J., Bækved-Hansen, M., Gudmundsson, O. O., Magnusson, S. H., Baldursson, G., . . . . Børglum, A. D. (2023). Genome-wide analyses of ADHD identify 27 risk loci, refine the genetic architecture and implicate several cognitive domains. Nature Genetics, 55 (2), 198-208.

### SUMMARY

The present disclosure entails a pharmacological solution by providing, in an embodiment, a embodi in a subject suffering from a dementia, including but not limited to Alzheimer's disease, Lewy body dementia, and Frontotemporal dementia, comprising administering to the subject a compound or pharmaceutically acceptable salt thereof that activates myocyte-specific enhancer factor 2 (MEF2) transcriptional activity.

In another embodiment, the present disclosure provides a method for treating Autism Spectrum Disorder (ASD) in a subject suffering therefrom, comprising administering to the subject a compound or pharmaceutically acceptable salt thereof that activates myocyte-specific enhancer factor 2 (MEF2) transcriptional activity.

In another embodiment, the present disclosure provides a method for treating Intellectual Disability (ID) optionally associated with ASD in a subject suffering therefrom, comprising administering to the subject a compound or pharmaceutically acceptable salt thereof that activates myocyte-specific enhancer factor 2 (MEF2) transcriptional activity.

Still another embodiment of the present disclosure is a method for treating a movement disorder in a subject suffering therefrom, comprising administering to the subject a compound or pharmaceutically acceptable salt thereof that activates myocyte-specific enhancer factor 2 (MEF2) transcriptional activity.

### BRIEF DESCRIPTION OF THE DRAWINGS

FIGS. 1A and 1B. Strategy and optimization of MEF2 screen. (FIG. 1A) Diagram of MEF2 and reporter construct used for screening for MEF2 activators. A MEF2-dependent firefly luciferase reporter gene was constructed by insertion of six tandem copies of the MEF2 responsive elements (MEF2 RE) from the brain creatine kinase enhancer upstream of the basal promoter of SV40. (FIG. 1B) Dose response results for Isoxazole 9, a compound that had previously been shown to activate MEF2 as a positive control, under different culture conditions. Different cells densities were used, and Z scores are shown for each cell density.

FIGS. 2A and 2B. Screening results for 50,000 compounds. Graphs showing results for each screening library by normalized luciferase intensity. Screening results are from three individual libraries performed in two batches. Both Bioactive and Biodiversity (A) and ReFRAME (B) libraries yielded active compounds. Y-axis also indicates level for a 5-fold increase in activity.

FIGS. 3A-3F. Dose response for hits in the primary screen. Dose-response plots for 6 exemplary compounds having the highest EC₅₀ and greater than 3-fold increase in MEF2 activity (A-F, respectively). Each plot shows Luciferase activity and the calculated EC₅₀. Each of these 6 compounds are from the ReFRAME library, which has already passed human toxicity testing.

FIG. 4. Dose-response for hits in the Renilla counterscreen. Dose response plots for AR-42, that had high EC₅₀ and >3-fold increase in MEF2 activity in the primary screen. (n=3).

FIGS. 5A and 5B. MEF2C activators increase MAP2 levels in MEF2C haploinsufficient (MHCS) patient hiPSC-derived cerebrocortical neurons. (FIG. 4A) MAP2 protein expression in neurons treated with AR-42 and (n=3 in 2 independent experiments). (FIG. 4B) Representative immunoblot of MAP2 expression and GAPDH loading control. Data represented as mean±SEM. * P<0.05 by one-way ANOVA and Tukey post-hoc test.

FIGS. 6A and 6B. AR-42 decreases hyperelectrical activity in MEF2C haploinsufficiency syndrome (MCHS) patient hiPSC-derived cerebrocortical neurons. (FIG. 5A) Representative traces of calcium events using fluo-4AM calcium-sensitive dye in hiPSC-derived neurons. MCHS neurons show increased calcium levels compared to WT neurons, but treatment with AR-42 normalizes this aberrant, excessive activity. (FIG. 5B) Quantification of calcium event frequency. Data represent mean±SEM. Sample size was 5 neurons imaged per coverslip in 3 coverslips. * P<0.05 by one-way ANOVA and Tukey post-hoc test.

FIG. 7. Mef2C-engineered human iPSC-derived neural progenitor cells (hNPCs) transplanted into the hippocampus rescue neurodegenerative pathology in Alzheimer's disease (AD) model. Laser scanning confocal microscopy of transplanted 3×Tg AD mice immunolabeled with antibodies against the dendritic neuronal marker MAP2 (upper panels) and synaptophysin Lower channels. Inset in top panels represent a higher magnification view of dendrites in the CA1 region. Image analysis shows that MAP2 pixel intensity is higher in the Mef2C group compared to vehicle and GFP groups. Similarly, levels of the synaptic marker synaptophysin were higher in the Mef2C group. Images in the inset represents the GFP tagged hNPC grafted into the hippocampus dentate (lines). n=3 per group, 1 way ANOVA with post hoc Fisher, p<0.05.

### DETAILED DESCRIPTION

The present disclosure is predicated, in part, upon the identification of small molecules that surprisingly activate MEF2 transcriptional activity and are thereby useful in halting or reversing the deficits in MEF2C activity found in ASD, movement disorders, and in diseases involving cognitive decline. As described in more detail below, the compounds emerged from a MEF2 screen of a library of compounds having, among other things, histories of clinical development and the advantage of demonstrated safety profiles.

### Embodiments

Embodiment 1. A method for treating cognitive decline in a subject suffering from a dementia, comprising administering to the subject a compound or pharmaceutically acceptable salt thereof that activates myocyte-specific enhancer factor 2 (MEF2) transcriptional activity.

Embodiment 2. The method according to embodiment 1, wherein the dementia is selected from Alzheimer's disease, Lewy body dementia, and Frontotemporal dementia.

Embodiment 3. The method according to embodiment 1 or 2, wherein the treating comprises slowing the cognitive decline.

Embodiment 4. The method according to embodiment 1 or 2, wherein the treating comprises arresting the cognitive decline.

Embodiment 5. The method according to embodiment 1 or 2, wherein the treating comprises reversing the cognitive decline.

Embodiment 6. A method for treating Autism Spectrum Disorder (ASD) in a subject suffering therefrom, comprising administering to the subject a compound or pharmaceutically acceptable salt thereof that activates myocyte-specific enhancer factor 2 (MEF2) transcriptional activity.

Embodiment 7. The method according to embodiment 6, wherein the subject exhibits at least one developmental condition selected from a social skill deficit, a communication deficit, a behavioral deficit, epilepsy, and combinations thereof.

Embodiment 8. The method according to embodiment 7, wherein the developmental condition is a social skill deficit.

Embodiment 9. The method according to embodiment 7, wherein the developmental condition is a communication deficit.

Embodiment 10. The method according to embodiment 7, wherein the developmental condition is a behavioral deficit.

Embodiment 11. A method for treating Intellectual Disability (ID) optionally associated with ASD in a subject suffering therefrom, comprising administering to the subject a compound or pharmaceutically acceptable salt thereof that activates myocyte-specific enhancer factor 2 (MEF2) transcriptional activity.

Embodiment 12. A method for treating MEF2C haploinsufficiency form of ASD/ID or ADHD in a subject suffering therefrom, comprising administering to the subject a compound or pharmaceutically acceptable salt thereof that activates myocyte-specific enhancer factor 2 (MEF2) transcriptional activity.

Embodiment 13. The method of embodiment 12, wherein the MEF2C haploinsufficiency form of ASD/ID is due to transposition, deletion, or other mutation of the MEF2C gene.

Embodiment 14. A method for treating a movement disorder in a subject suffering therefrom, comprising administering to the subject a compound or pharmaceutically acceptable salt thereof that activates myocyte-specific enhancer factor 2 (MEF2) transcriptional activity.

Embodiment 15. The method according to embodiment 4, wherein the movement disorder is Parkinson's disease (PD).

Embodiment 16. The method according to embodiment 4, wherein the movement disorder is parkinsonism.

Embodiment 17. The method according to embodiment 16, wherein the parkinsonism is idiopathic PD.

Embodiment 18. The method according to embodiment 16 or 17, wherein the parkinsonism is a sign or symptom selected from bradykinesia, rigidity, and postural instability.

Embodiment 19. The method according to embodiment 6, wherein the parkinsonism is atypical parkinsonism.

Embodiment 20. The method according to embodiment 16 or 17, wherein the parkinsonism is selected from multiple system atrophy, progressive supranuclear palsy, corticobasal syndrome, dementia with Lewy bodies, drug-induced parkinsonism, and vascular parkinsonism.

Embodiment 21. A method for treating MEF2C dysfunction in a subject suffering from attention deficit and hyperactivity disorder (ADHD), comprising administering to the subject a compound or pharmaceutically acceptable salt thereof that activates myocyte-specific enhancer factor 2 (MEF2) transcriptional activity.

Embodiment 22. The method according to any of embodiments 1 to 21, wherein the compound or pharmaceutically acceptable salt thereof is one selected from Table 1.

Embodiment 22. The method according to any of embodiments 1 to 22, wherein the compound is AR-42:

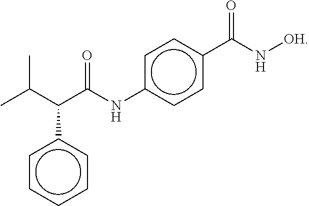

### Definitions

Some compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, and/or tautomer thereof. Thus, for instance, a compound of the present disclosure includes a pharmaceutically acceptable salt of a tautomer of the compound.

In this description, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this example, the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Methods of Use

In some embodiments, the small molecule activator of MEF2 transcriptional activity is administered to a subject suffering from cognitive decline. Cognitive decline can be manifested in various pathologies, including Alzheimer's disease, Lewy body dementia, and Frontotemporal dementia. In some embodiments, treatment comprises the slowing of cognitive decline in the subject. In other embodiments, treatment comprises temporary or indefinite arrest of the cognitive decline. In still other embodiments, treatment comprises reversal of the cognitive decline.

The cognitive decline can be manifested in a relatively early stage, such as in a subject suffering from mild cognitive impairment. Cognitive decline can be manifested in subjects in later stages characterized, in part, by the presence of dementia. Cognitive decline in subjects can be characterized by the presence of one or more conditions including memory loss, language challenges such as difficulty in choice of words and/or understanding verbal or written communications, loss of attention or focus, challenges in exercising reasoning and judgment, and struggles in complex decision-making, including the execution of everyday tasks. Treatment of the cognitive decline in accordance with the methods described herein can be assessed by observance in the change of any one or more the conditions during the treatment period, including the lessening in intensity, frequency, severity, acuity, or even disappearance of any of the conditions.

In other embodiments, the present disclosure provides a method for treating Autism Spectrum Disorder (ASD) in a subject suffering therefrom, comprising administering a compound as disclosed herein. ASD is manifested variously in subjects in a range of behavioral or developmental conditions and corresponding severities. A subject suffering from ASD can exhibit one or more conditions including social skill deficit, communication deficit, and behavioral deficit. Specific examples include but are not limited to delayed language skills, delayed movement skills, delayed cognitive or learning skills, hyperactivity, impulsivity, inattentive behavior, epilepsy or seizure disorder, unusual sleeping or eating habits, difficulty in social communication and interaction with others, restricted interests, repetitive behaviors. Although the causes of ASD are yet unknown, the method of the present disclosure can ameliorate one or more of these conditions in a subject who is diagnosed with ASD, such as in accordance with the Diagnostic and Statistical Manual of Mental Disorders (DSM-5). A subject can include a young child, such as one who is 9-, 18-, 24-, or 30-months old, an older child or adolescent, and an adult.

In still additional embodiments, the present disclosure provides a method for treating Intellectual Disability (ID) that is optionally associated with ASD in a subject suffering therefrom, comprising administering to the subject a compound as disclosed herein. Subjects who meet criteria for a diagnosis of AD often, but not always, also meet criteria for ID. The present methods are useful in treating the subjects with ID, whether the ID is co-occurring with ASD. The clinician can perform routine screening to make a diagnostic assessment of the subject in order to determine whether ID and ASD co-occur. An advantage of the methods disclosed herein is their treatment of a range of symptoms and conditions, including ID. ID is often manifested by deficits in learning, memory, and other cognitive spheres. ID includes deficits in both intellectual functioning and in adaptive behavior, which covers many everyday social and practical skills, with onset generally occurring prior to the age of the early 20's.

In additional embodiments, the present disclosure provides a method for treating a movement disorder in a subject suffering therefrom. The method comprising administering to the subject a compound as disclosed herein. In some embodiments, the movement disorder is Parkinson's disease (PD).

In other embodiments, the movement disorder is parkinsonism. As used herein, the term “parkinsonism” encompasses a collection of signs and symptoms that can characterize PD, but may arise from conditions other than, and thereby mimic, PD. Parkinsonism includes, in various embodiments, idiopathic PD, which is the most common form of Parkinsonism. Examples of idiopathic PD include slowness (bradykinesia), stiffness (rigidity), and tremor and imbalance (postural instability).

In other embodiments, the parkinsonism is atypical parkinsonism or atypical parkinsonism disorders, and these are typically represented in a minority of subjects who present with symptoms suggestive of PD. Atypical parkinsonism disorders include, for example, multiple system atrophy (MSA), progressive supranuclear palsy (PSP), corticobasal syndrome (CBS), dementia with Lewy bodies (DLB), drug-induced parkinsonism, and vascular parkinsonism (VP). MSA refers to a collection of neurodegenerative disorders. Symptoms of MSA include incoordination (ataxia), dysfunction in the autonomic nervous system that automatically controls functions such as blood pressure and bladder function. PSP is the most common degenerative embodiment of atypical parkinsonism, characterized by symptoms that progress more rapidly than PD, including propensity to falling, limitations in eye movements, dysphagia, dysarthria, sleep problems, and dementia. CBS is the least common atypical parkinsonism, usually manifesting symptoms first in one limb, and these can include dystonia in the affected limb, myoclonus, apraxia, and aphasia. DLB is generally characterized as arising from abnormal deposits alpha-synuclein in multiple areas of the brain, with symptoms including progressive dementia and hallucinations, which can be joined in later stages by symptoms of parkinsonism. Drug-induced parkinsonism, also known as the most common embodiment of secondary parkinsonism, is characterized by tremors and/or postural instability as side effects of drugs including antipsychotics, some antiemetics, some antidepressants, reserpine, tetrabenazine, and some calcium channel blockers. VP is characterized by multiple small strokes in the brain that can manifest as severe onset of parkinsonism following a stroke, and/or by evidence of vascular disease in the brain in combination with deterioration, early cognitive difficulties, and lower body problems such as gait and balance difficulties.

### Activators of MEF2 Transcriptional Activity

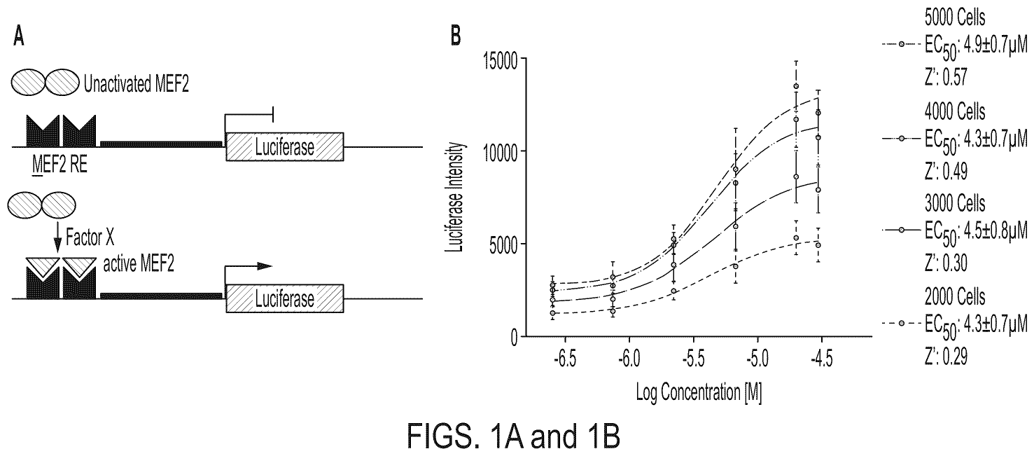

Compounds that are useful in the methods disclosed herein were identified from a screen of a compound library as described in more detail below. Human neural progenitor cells were transfected with a Luciferase-MEF2 reporter plasmid using an electroporation method (FIG. 1A). The cells were then plated using a robotic platform on 1536-well plates. A positive compound, Isoxazole 9, that was previously identified in a small screen was used for the assay optimization. Cell density and transfection conditions were optimized to attain robust Z scores (FIG. 1B).

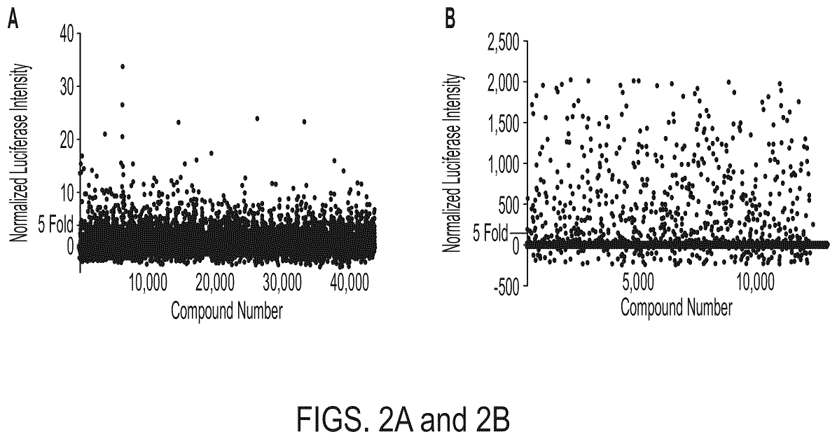

The screen sampled over 50,000 compounds: 13,000 compounds were from the ReFRAME library, which is a CALIBR and Scripps research library of compounds that have reached clinical development, FDA- or EMA-approval, or undergone significant preclinical profiling with demonstrated safety profiles. 43,000 compounds were from the BioActive and BioDiversity libraries. All compounds were screened at a concentration of 10 μM to identify compounds that upregulate MEF2 transcriptional activity (FIGS. 2A and 2B). In further embodiments of the present disclosure, Table 1 illustrates such compounds, sources, and their respective concentrations for half-maximal activity derived from the hill equation model (qAC50).

TABLE 1
Illustrative Compounds from MEF2 Transcriptional Activity Screen

| Structure | Source | Name | qAC₅₀ [M] |
|---|---|---|---|
| 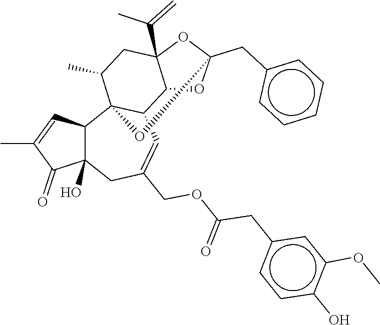 | BIOTREND Chemicals | Resiniferatoxin | 5.112E−9 |
| 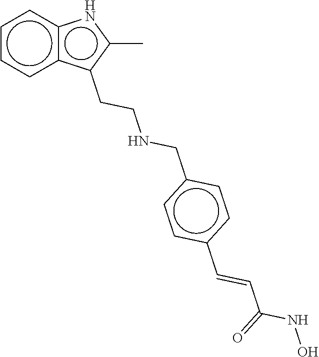 | MedChem Express | Panobinostat lactate | 1.219E−8 |
| 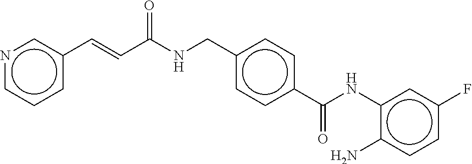 | Astatech | Chidamide | 3.665E−8 |
| 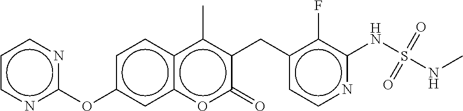 | Pharmaron | RO-5126766 | 3.906E−8 |
| 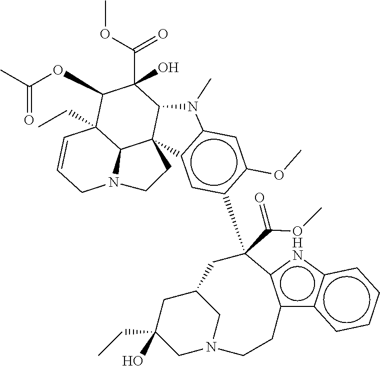 | Enamine | Vinblastine sulfate | 6.733E−8 |
| 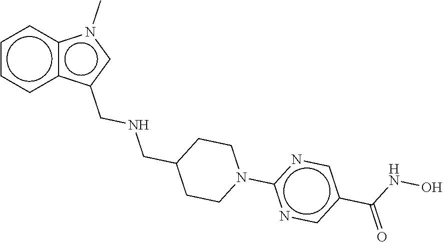 | MedChem Express | quisinostat | 9.067E−8 |
| 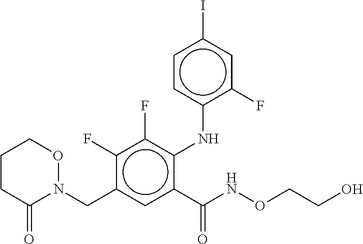 | MedChem Express | CH-4987655 | 1.074E−7 |
| 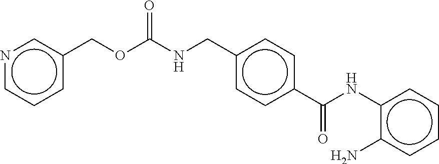 | AK Scientific | entinostat | 1.490E−7 |
| 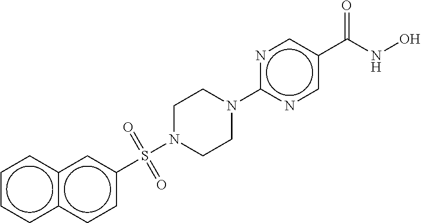 | Advanced ChemBlocks Inc | JNJ-16241199 | 1.588E−7 |
| 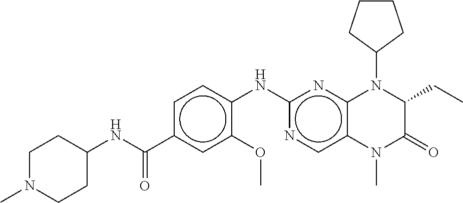 | Adooq Bioscience | BI-2536 | 2.091E−7 |
| 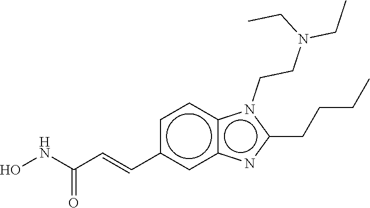 | Lancrix Chemicals | pracinostat | 2.096E−7 |
| 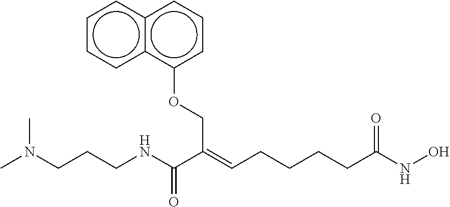 | WuXi | CG-200745 | 2.143E−7 |
| 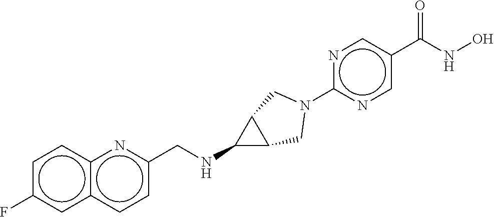 | Chempartner | CHR-3996 | 2.795E−7 |
| 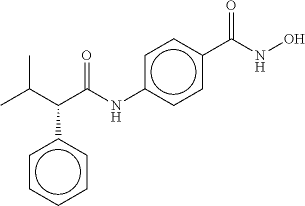 | MedChem Express | AR-42 | 4.385E−7 |
| 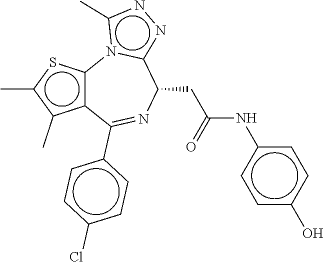 | Selleck Chemicals | OTX-015 | 4.505E−7 |
| 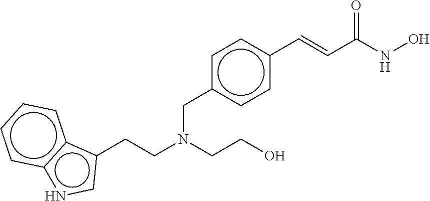 | Shanghai iChemical Technology Co., Ltd | LAQ 824 | 5.299E−7 |
| 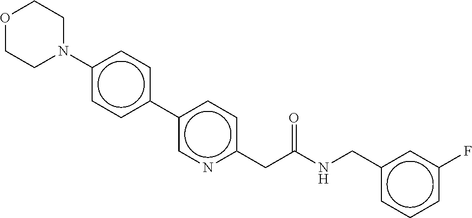 | Chempartner | KX-02 | 5.798E−7 |
| 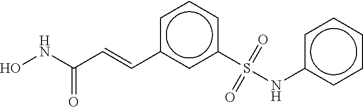 | MedChem Express | belinostat | 6.701E−7 |
| 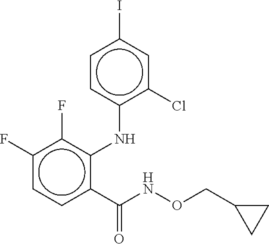 | Lancrix Chemicals | CI-1040 | 8.536E−7 |
| 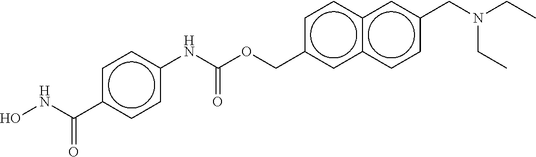 | WuXi | Givinostat hydrochloride | 9.719E−7 |
| 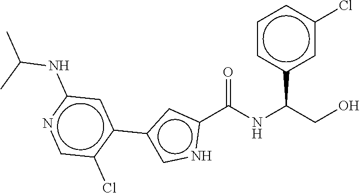 | CEG Chemical Science & Technology Co., Ltd. | BVD-523 | 9.870E−7 |
| 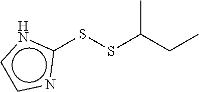 | MedChem Express | PX-12 | 9.947E−7 |
| 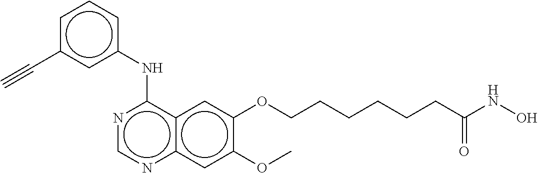 | AK Scientific | CUDC-101 | 1.051E−6 |
| 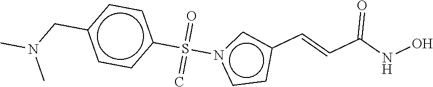 | Adooq Bioscience | resminostat | 1.233E−6 |
| 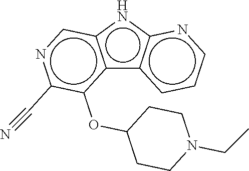 | WuXi | GDC-0425 | 1.257E−6 |
| 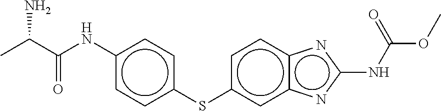 | Chempartner | DENIBULIN | 1.514E−6 |
| 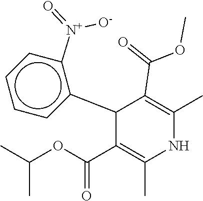 | Specs | | 1.534E−6 |
| 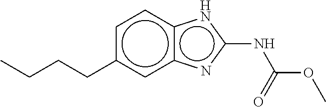 | Sigma- Aldrich | PARBENDAZOLE | 1.746E−6 |
| 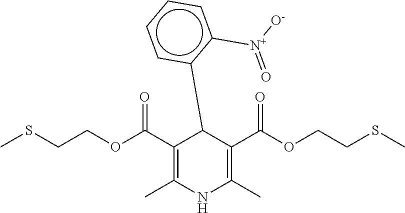 | Specs | | 2.071E−6 |
| 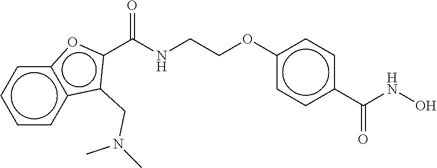 | MedChem Express | abexinostat | 2.198E−6 |
| 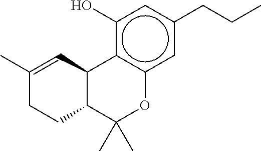 | WuXi | DELTA-9- TETRA- HYDROCANNA BIVARIN | 2.290E−6 |
| 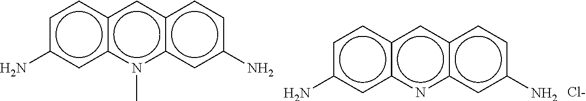 | Chem-Impex | Acriflavine | 2.314E−6 |
| 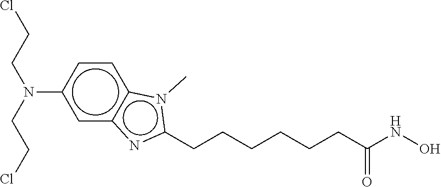 | eNovation Chemicals LLC | CY-190602 | 2.412E−6 |
| 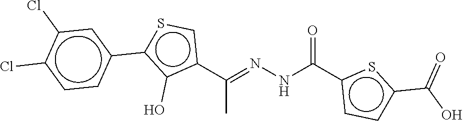 | Chempartner | ONO-7746 | 2.571E−6 |
| 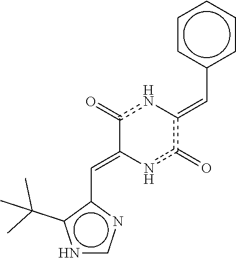 | MedChem Express | plinabulin | 2.673E−6 |
| 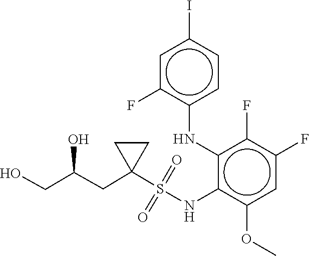 | Cayman Chemical | Refametinib | 2.731E−6 |
| 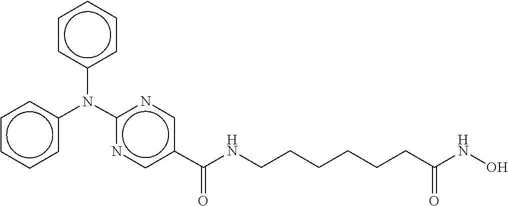 | MedChem Express | ricolinostat | 2.787E−6 |
| 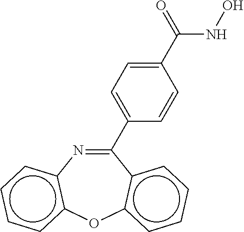 | Pharmaron | EVP-0334 | 3.025E−6 |
| 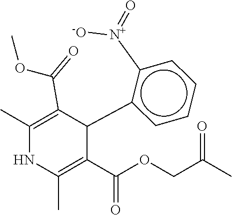 | WuXi | ARANIDIPINE | 3.105E−6 |
| 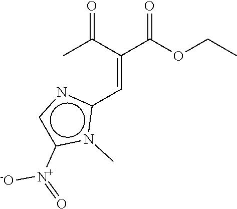 | WuXi | Propenidazole | 3.110E−6 |
| 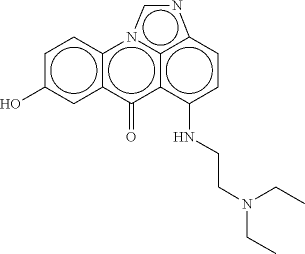 | Sigma- Aldrich | Imidazoacridinone | 3.661E−6 |
| 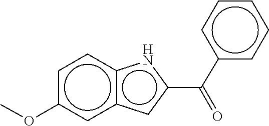 | MedChem Express | D-64131 | 3.741E−6 |
| 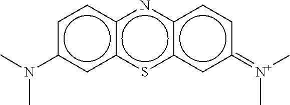 | TCI Europe | methylthioninium chloride | 3.769E−6 |
| 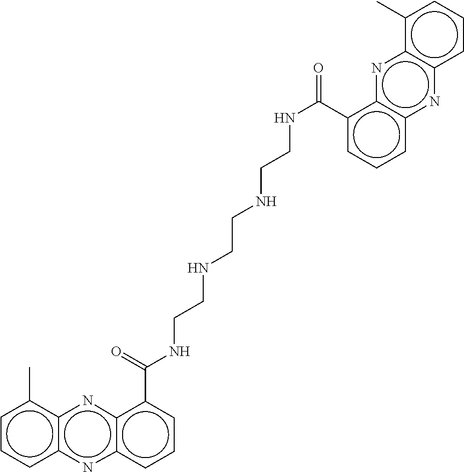 | Chempartner | XR 5944 | 4.519E−6 |
| 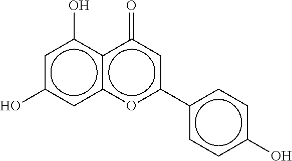 | Sigma- Aldrich | APIGENIN | 4.688E−6 |
| 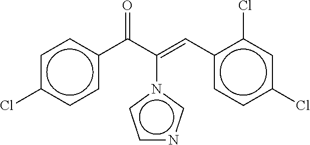 | WuXi | TS-80 | >4.970E−6 |
| 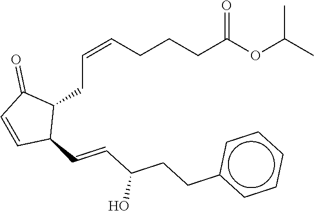 | Pharmaron | Posaraprost | >4.970E−6 |
| 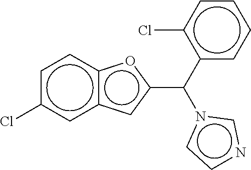 | Pharmaron | BECLICONAZOLE | 5.097E−6 |
| 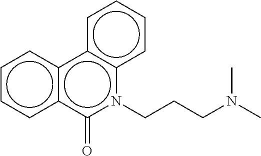 | Ark Pharm, Inc | FANTRIDONE | 5.381E−6 |
| 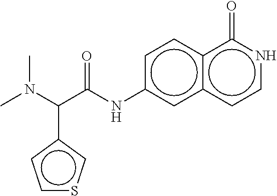 | Jubilant Chemsys | Verosudil | 7.537E−6 |
| 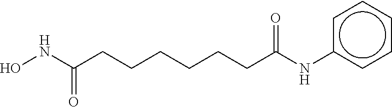 | Cayman Chemical | vorinostat | 7.812E−6 |
| 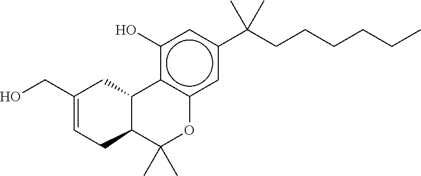 | Tocris | Dexanabinol | 7.971E−6 |
| 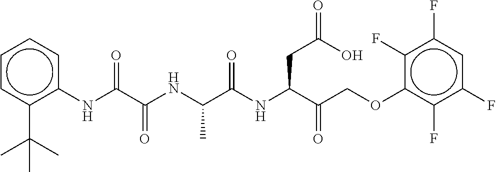 | MedChem Express | emricasan | 8.708E−6 |
| 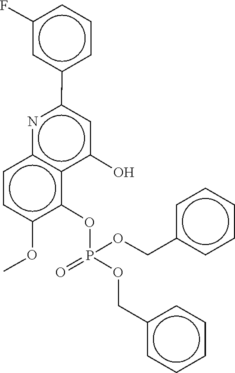 | Ark Pharm, Inc | TRX-818 | 1.005E−5 |
| 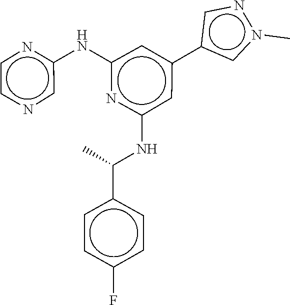 | Chempartner | NS-018 | 1.124E−5 |
| 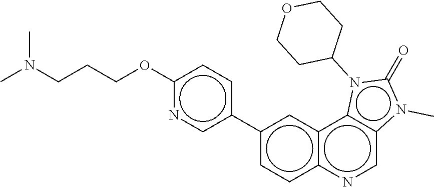 | MedChem Express | AZD-0156 | 1.176E−5 |
| 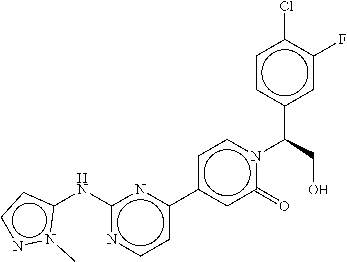 | Selleck Chemicals | RG-7842 | 1.232E−5 |
| 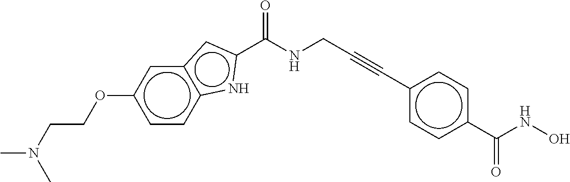 | WuXi | CRA-026440 | 1.288E−5 |
| 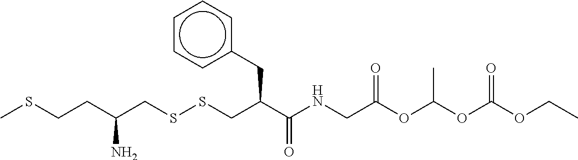 | Chempartner | PL-37 | 1.290E−5 |
| 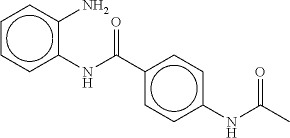 | MedChem Express | Tacedinaline | 1.306E−5 |
| 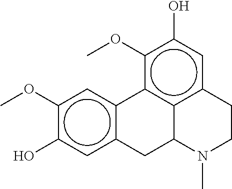 | Sigma- Aldrich | boldine | 1.326E−5 |
| 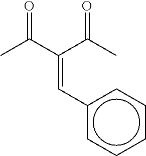 | Sigma- Aldrich | | 1.570E−5 |
| 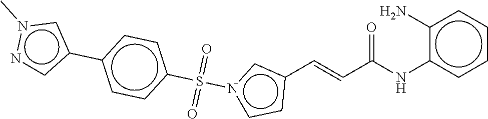 | Selleck Chemicals | 4SC-202 | 1.754E−5 |
| 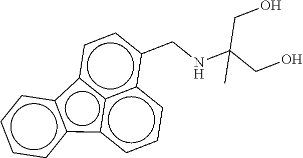 | Chempartner | 773U82 | 1.888E−5 |
| 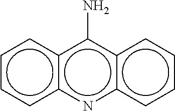 | Sigma- Aldrich | ACRISORCIN | 1.927E−5 |
| 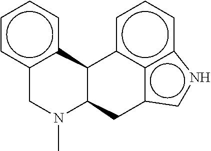 | Bio-Techne | CY-208-243 | 1.955E−5 |
| 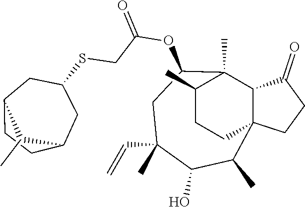 [SMILES: C=C[C@]1(C)C[C@@H](OC(=O)CS[C@@H]2C[C@H]3CC[C@@H](C2)C3C)[C@]2(C)[C@H](C)CC[C@]3(CCC(=O)[C@H]32)[C@@H](C)[C@@H]1O] | Sigma- Aldrich | retapamulin | 1.961E−5 |
| 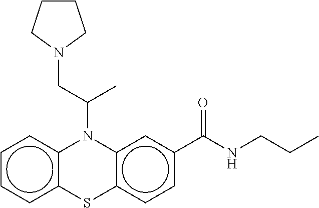 | Chempartner | Apadoline | 1.966E−5 |
| 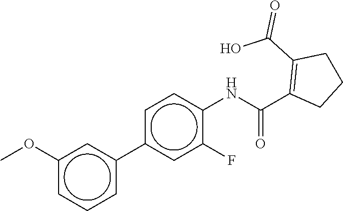 | ATOMOLE SCIENTIFI C CO., LTD. | vidofludimus | >1.990E−5 |
| 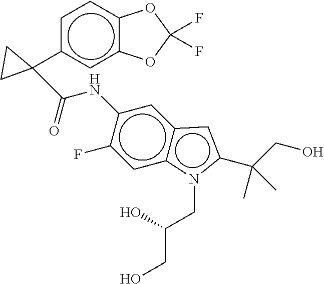 | Selleck Chemicals | VX-661 | >1.990E−5 |
| 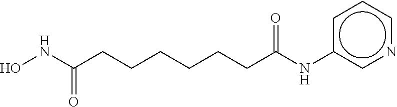 | Shanghai iChemical Technology Co., Ltd | pyroxamide | >1.990E−5 |
| 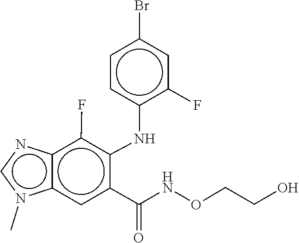 | MedChem Express | binimetinib | >1.990E−5 |
| 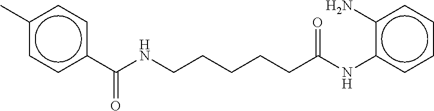 | MedChem Express | RG-2833 | >1.990E−5 |
| 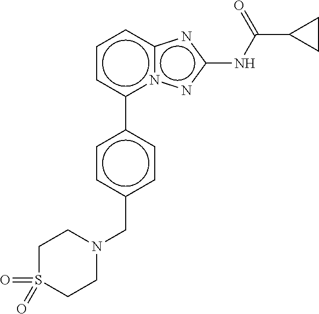 | MedChem Express | Filgotinib | >1.990E−5 |
| 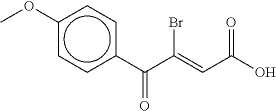 | Sigma- Aldrich | Bromebrate sodium | >1.990E−5 |
| 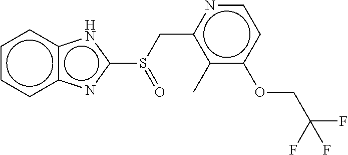 | Sigma- Aldrich | dexlansoprazole | >1.990E−5 |
| 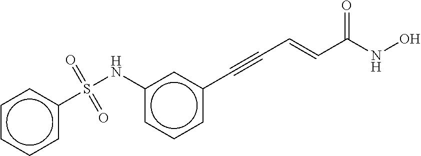 | Sigma- Aldrich | OXAMFLATIN | >1.990E−5 |
| 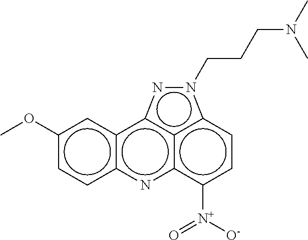 | Sigma- Aldrich | Pyrazoloacridine | >1.990E−5 |
| 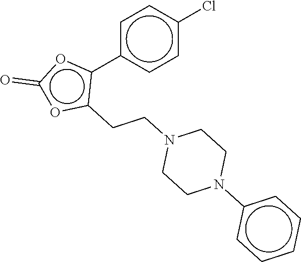 | Chempartner | CLODOXOPONE | >1.990E−5 |
| 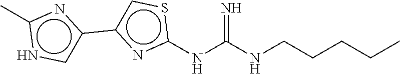 | WuXi | CP 66948 | >1.990E−5 |
| 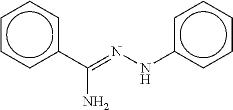 | Vitas-M | CBS-1114 | >1.990E−5 |
| 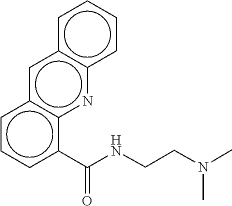 | AMS Private Supplier 1 | SN-22995 | >1.990E−5 |
| 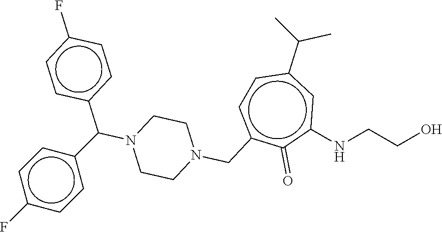 | Chempartner | U 92032 | >1.990E−5 |
| 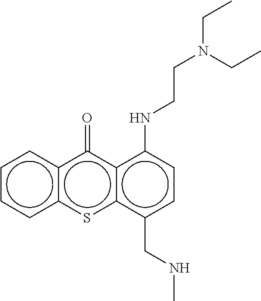 | WuXi | WIN 33156 | >1.990E−5 |
| 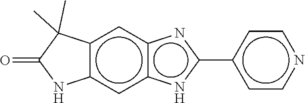 | Chempartner | ADIBENDAN | >1.990E−5 |
| 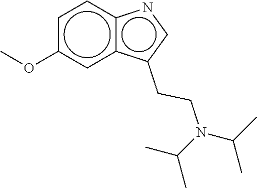 | Whhan Daybiochem Technology | 5-MeO-DIPT | >1.990E−5 |
| 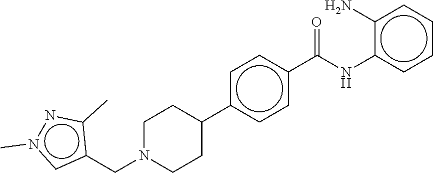 | Chempartner | AZD-9468 | >1.990E−5 |
| 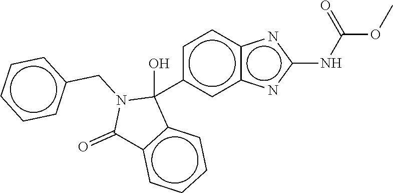 | WuXi | BMS-908662 | >1.990E−5 |

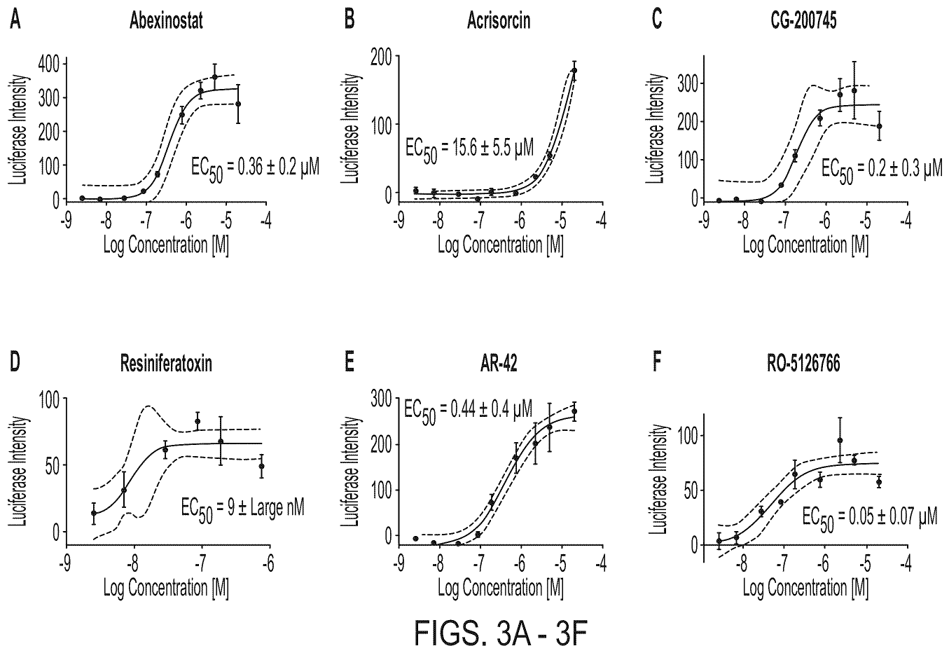

The initial (primary screen) hits were then confirmed in a secondary screen with at least 5 hits that were likely to have CNS permeability and that activate MEF2 reporter gene activity by at least 3-fold at less than about 10 micromolar concentrations. Thus, the secondary screen was performed on the top 175 hits, with a cut-off based on the robust Z-score of 5 SD above the control, representing at least a 6.8-fold change, which is actually greater than the minimal criteria of 3-fold stated above. In this screen was observed a dose-response effect, and there was identified at least 20 compounds with an EC₅₀ of less than 10 μM and at least a 3-fold increase in MEF2 reporter activity; the top 6 compounds were then identified via CNS permeability analysis (FIGS. 3A-3F).

At least two compounds from the secondary screen were identified as having excellent CNS permeability as indicated, for example, by a Parallel Artificial Membrane Permeability Assay (PAMPA) value of ≥2×10⁻⁶ cm s⁻¹. As summarized in Table 2 below, both compounds RO-512766 (Hyohdoh, I. et al., ACS Med Chem Lett 4 (2013) 1059-1063) and Acrisorcin (Svobodova, B. et al., Biomolecules 9 (8) (2019) 379) (FIGS. 3B and 3F) have a PAMPA value of 6 as well as other metrics that predict excellent CNS permeability, e.g., based on cLogP, polar surface area and PAMPA values (Rankovic, Z., J Med Chem 58 (2015) 2584-2608).

TABLE 2
Illustrative Compounds that are blood brain barrier (BBB) permeable.

| | | | | | | PAMPA |
|---|---|---|---|---|---|---|
| | Molecular | | | | Total Polar | Pₑ |
| | Weight | | Hydrogen- | Hydrogen- | Surface | (10⁻⁶ cm |
| Compound | (g/mol) | cLogP | Acceptors | Donors | Area (Å²) | s⁻¹) |
| Resiniferatoxin | 628.716 | 5.4 | 9 | 2 | 120.75 | |
| CG-200745 | 427.543 | 3.3 | 7 | 3 | 90.9 | |
| Abexinostat | 397.43 | 1.6 | 8 | 3 | 104.04 | |
| AR-42 | 312.368 | 2.8 | 5 | 3 | 78.43 | |
| RO-5126766 | 471.468 | 1.9 | 10 | 2 | 140.78 | 6 |
| Acrisorcin | 194.236 | 2.6 | 2 | 1 | 38.91 | 6 |

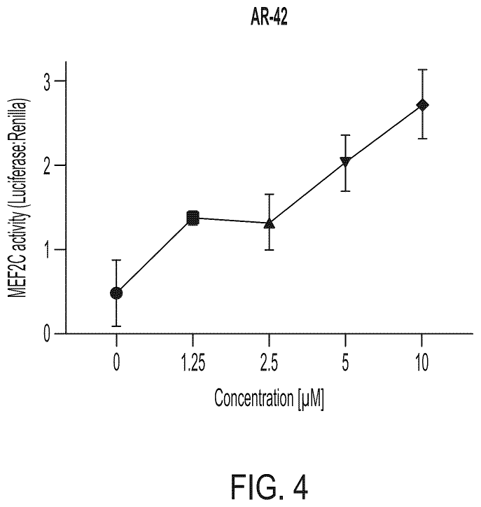

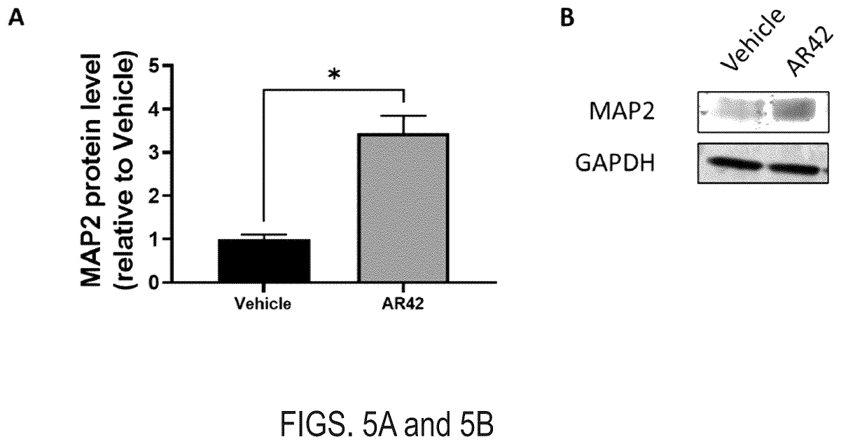

In an embodiment, a compound for use in a method as described herein is AR-42 (Table 1). To illustrate, a confirmatory screen and a counter-screen with AR-42 showed increased MEF2 transcriptional activity even when corrected with control Renilla-based (non-specific) reporter fluorescence (FIG. 4). AR-42 demonstrated utility that is relevant to the E/I (excitatory/inhibitory) imbalance that has been observed in Autism Spectrum Disorder (ASD) brain and in the Alzheimer's disease (AD) brain (S. Ghatak S, et al., Annu Rev Pharm Toxicol 61 (2021) 701-721). Specifically, AR-42 decreased aberrant hyperactivity, as reflected by elevated Ca²⁺ signaling, in hiPSC-derived cerebrocortical neurons bearing a MEF2C haploinsufficient-causing mutation (a cause of severe ASD and Intellectual Disability [ID]) against isogenic WT control (FIGS. 5A and 5B).

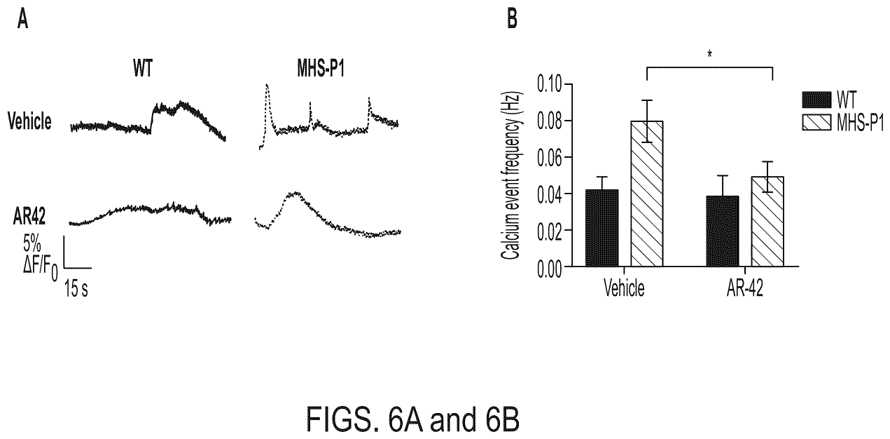

Additionally, the neurons of patients with MEF2C haploinsufficiency syndrome (MCHS) display shorter neurites. The neuronal marker MAP2 was monitored as a biomarker for neurites in the MCHS hiPSC-derived neurons versus WT control: compromised levels of MAP2 were found, but they were corrected by treatment with AR-42 in vitro (FIGS. 6A and 6B). These types of assays correlate well with improvement in behavioral studies in this type of ASD/ID, as illustrated by the use mouse models of MCHS (S. Tu (2017)).

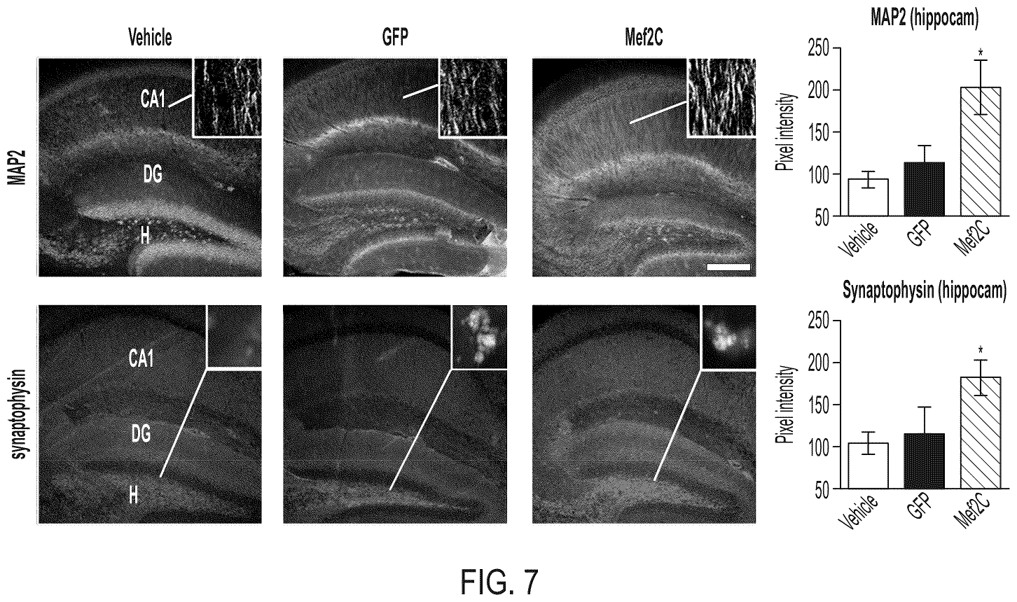

Further investigated was the role of MEF2C in Alzheimer's disease (AD). In brief, when MEF2C-engineered hiPSC-derived neural progenitor cells (hNPCs) were transplanted into the hippocampus of 3×Tg AD mouse model, it was found that there were increased synapses and neurite markers in the endogenous cells, genetic proof of a noncell autonomous trophic effect of MEF2C in these older AD mice (FIG. 7) and generally as further genetic proof of activated MEF2C in dementia models.

### Pharmaceutical Compositions

The disclosure also provides a pharmaceutical composition comprising a therapeutically effective amount of one or more compounds as described herein, or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

In one embodiment, the pharmaceutical composition comprises a compound selected from those illustrated in any table disclosed herein or a pharmaceutically acceptable salt, stereoisomer, and/or tautomer thereof, and a pharmaceutically acceptable carrier.

The pharmaceutical composition of the present disclosure is formulated, dosed, and administered in a fashion consistent with good medical practice. Factors for consideration in this context include the particular disorder being treated, the particular subject being treated, the clinical condition of the subject, the cause of the disorder, the site of delivery of the agent, the method of administration, the scheduling of administration, and other factors known to medical practitioners.

The “therapeutically effective amount” of a compound or a pharmaceutically acceptable salt, stereoisomer, and/or tautomer thereof that is administered is governed by such considerations, and is the minimum amount necessary to activate or increase MEF2 transcriptional activity. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.01 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In any of the foregoing embodiments the dosage form can be administered once a day or twice per day.

The compositions of the present disclosure can be administered orally, topically, parenterally, by inhalation or spray or rectally in dosage unit formulations. The term parenteral as used herein includes subcutaneous injections, intravenous, intramuscular, intracisternal injection, perispinal injection, or infusion techniques.

Suitable oral compositions as described herein include without limitation tablets, troches, lozenges, aqueous or oily suspensions, dispersible powders or granules, emulsion, hard or soft capsules, syrups or elixirs.

In another aspect, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of the compound.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

The compounds of the present disclosure may also be administered in the form of suppositories for rectal administration. These compositions can be prepared by mixing the compounds with a suitable non-irritating excipient which is solid at ordinary temperatures but liquid at the rectal temperature and will therefore melt in the rectum to release the compound. Such materials are cocoa butter and polyethylene glycols.

Compositions for parenteral administrations are administered in a sterile medium. Depending on the vehicle used and concentration the concentration of the compounds in the formulation, the parenteral formulation can either be a suspension or a solution containing dissolved compound. Adjuvants such as local anesthetics, preservatives and buffering agents can also be added to parenteral compositions.

We claim:

1. A method for treating cognitive decline in a subject suffering from a dementia, comprising administering to the subject a compound or pharmaceutically acceptable salt thereof that activates myocyte-specific enhancer factor 2 (MEF2) transcriptional activity.

2. The method according to claim 1, wherein the dementia is selected from Alzheimer's disease, Lewy body dementia, and Frontotemporal dementia.

3. The method according to claim 1 or 2, wherein the treating comprises slowing the cognitive decline.

4. The method according to claim 1 or 2, wherein the treating comprises arresting the cognitive decline.

5. The method according to claim 1 or 2, wherein the treating comprises reversing the cognitive decline.

6. A method for treating Autism Spectrum Disorder (ASD) in a subject suffering therefrom, comprising administering to the subject a compound or pharmaceutically acceptable salt thereof that activates myocyte-specific enhancer factor 2 (MEF2) transcriptional activity.

7. The method according to claim 6, wherein the subject exhibits at least one developmental condition selected from a social skill deficit, a communication deficit, a behavioral deficit, epilepsy, and combinations thereof.

8. The method according to claim 7, wherein the developmental condition is a social skill deficit.

9. The method according to claim 7, wherein the developmental condition is a communication deficit.

10. The method according to claim 7, wherein the developmental condition is a behavioral deficit.

11. A method for treating Intellectual Disability (ID) optionally associated with ASD in a subject suffering therefrom, comprising administering to the subject a compound or pharmaceutically acceptable salt thereof that activates myocyte-specific enhancer factor 2 (MEF2) transcriptional activity.

12. A method for treating MEF2C haploinsufficiency form of ASD/ID in a subject suffering therefrom, comprising administering to the subject a compound or pharmaceutically acceptable salt thereof that activates myocyte-specific enhancer factor 2 (MEF2) transcriptional activity.

13. The method of claim 12, wherein the MEF2C haploinsufficiency form of ASD/ID is due to transposition, deletion, or other mutation of the MEF2C gene.

14. A method for treating a movement disorder in a subject suffering therefrom, comprising administering to the subject a compound or pharmaceutically acceptable salt thereof that activates myocyte-specific enhancer factor 2 (MEF2) transcriptional activity.

15. The method according to claim 4, wherein the movement disorder is Parkinson's disease (PD).

16. The method according to claim 4, wherein the movement disorder is parkinsonism.

17. The method according to claim 16, wherein the parkinsonism is idiopathic PD.

18. The method according to claim 16 or 17, wherein the parkinsonism is a sign or symptom selected from bradykinesia, rigidity, and postural instability.

19. The method according to claim 16, wherein the parkinsonism is atypical parkinsonism.

20. The method according to claim 16 or 17, wherein the parkinsonism is selected from multiple system atrophy, progressive supranuclear palsy, corticobasal syndrome, dementia with Lewy bodies, drug-induced parkinsonism, and vascular parkinsonism.

21. A method for treating MEF2C dysfunction in a subject suffering from attention deficit and hyperactivity disorder (ADHD), comprising administering to the subject a compound or pharmaceutically acceptable salt thereof that activates myocyte-specific enhancer factor 2 (MEF2) transcriptional activity.

22. The method according to any of claims 1 to 21, wherein the compound or pharmaceutically acceptable salt thereof is one selected from the following table:

| Structure | Name |
|---|---|
| 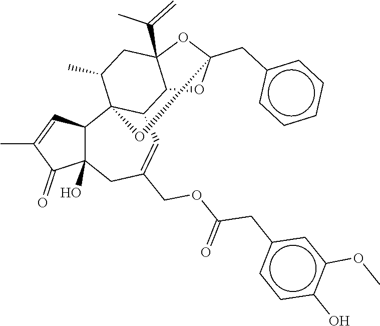 | Resiniferatoxin |
| 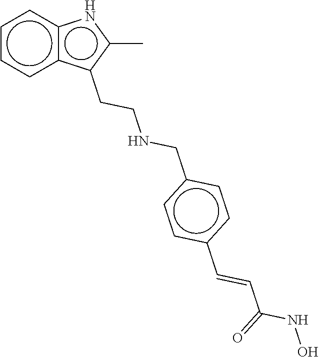 | Panobinostat lactate |
| 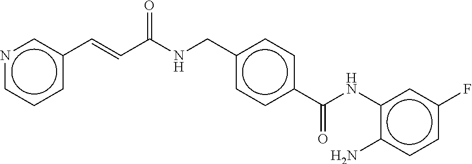 | Chidamide |
| 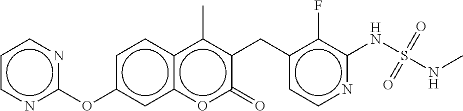 | RO-5126766 |
| 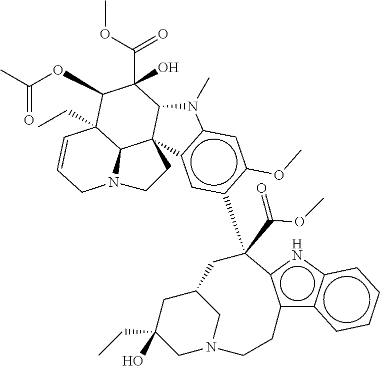 | Vinblastine sulfate |
| 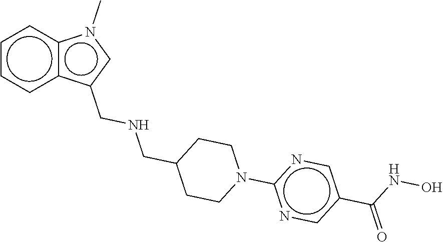 | quisinostat |
| 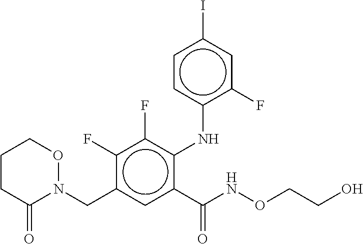 | CH-4987655 |
| 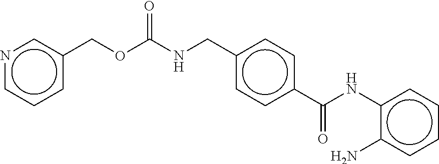 | entinostat |
| 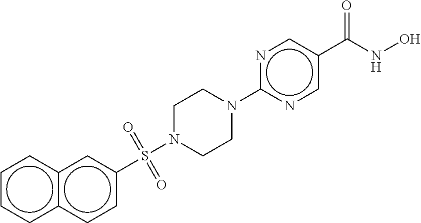 | JNJ-16241199 |
| 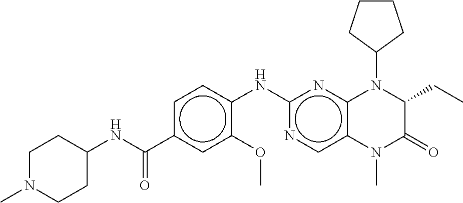 | BI-2536 |
| 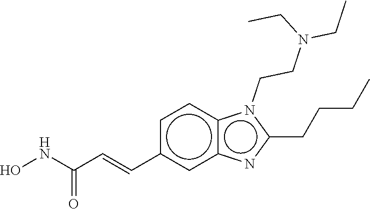 | pracinostat |
| 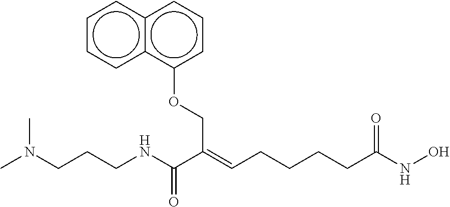 | CG-200745 |
| 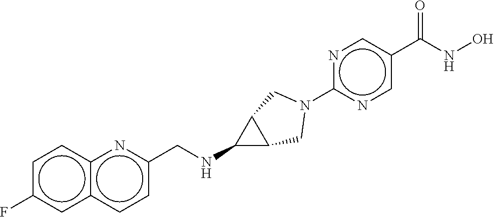 | CHR-3996 |
| 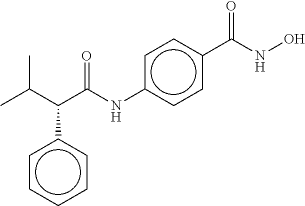 | AR-42 |
| 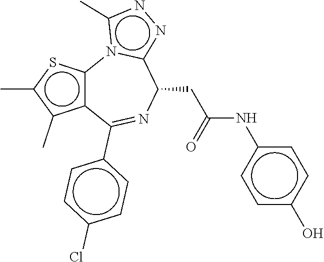 | OTX-015 |
| 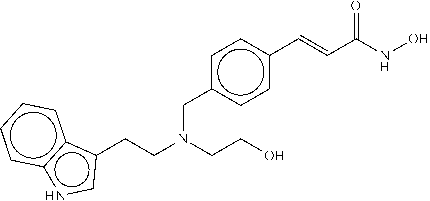 | LAQ 824 |
| 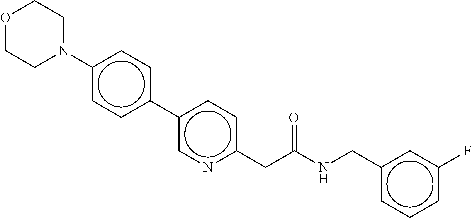 | KX-02 |
| 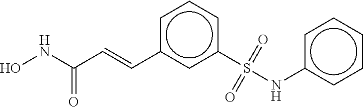 | belinostat |
| 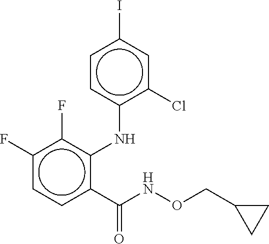 | CI-1040 |
| 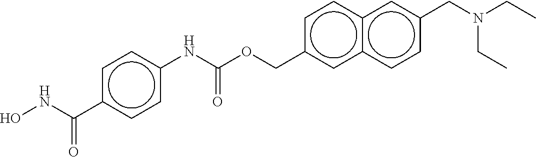 | Givinostat hydrochloride |
| 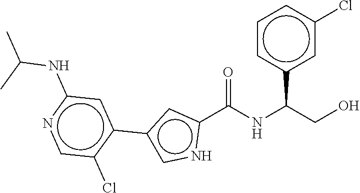 | BVD-523 |
| 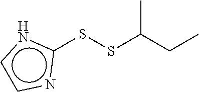 | PX-12 |
| 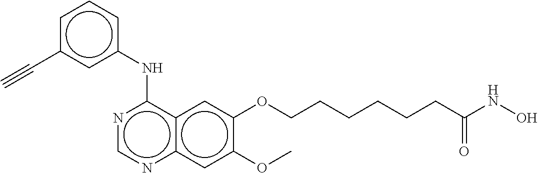 | CUDC-101 |
| 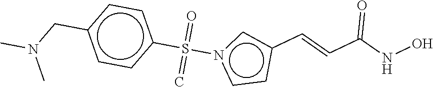 | resminostat |
| 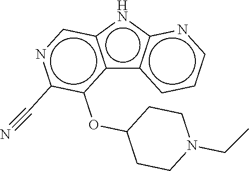 | GDC-0425 |
| 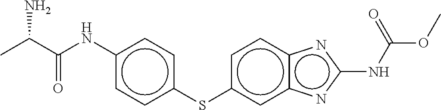 | DENIBULIN |
| 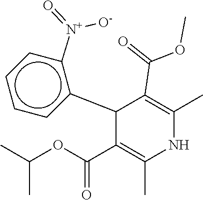 | |
| 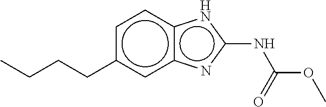 | PARBENDAZOLE |
| 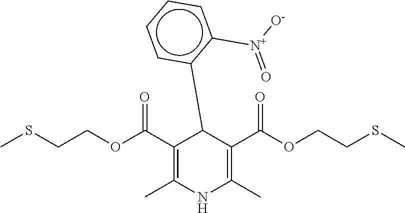 | |
| 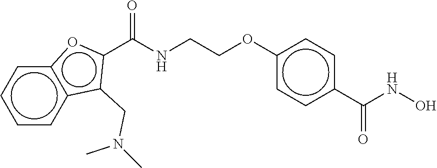 | abexinostat |
| 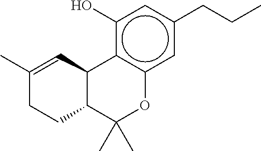 | DELTA-9- TETRAHYDROCANNA BIVARIN |
| 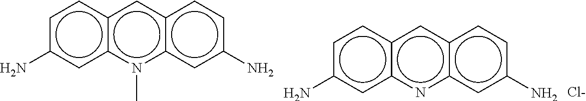 | Acriflavine |
| 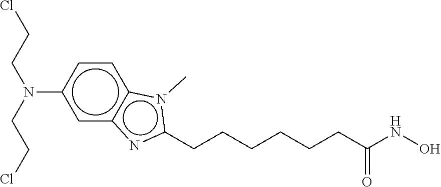 | CY-190602 |
| 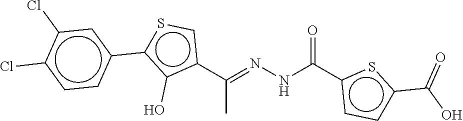 | ONO-7746 |
| 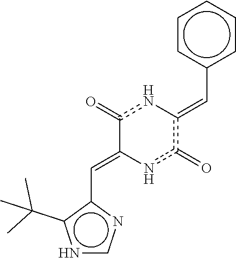 | plinabulin |
| 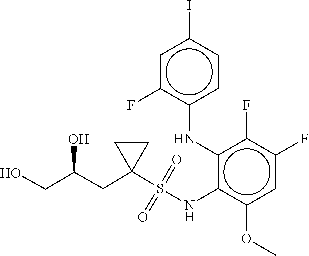 | Refametinib |
| 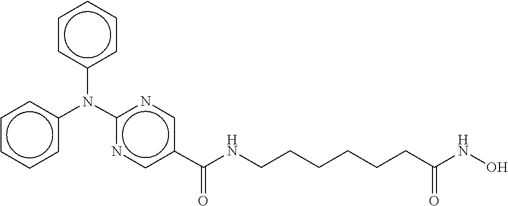 | ricolinostat |
| 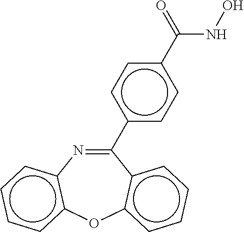 | EVP-0334 |
| 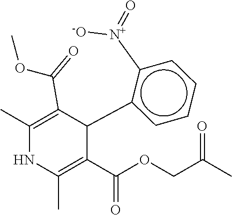 | ARANIDIPINE |
| 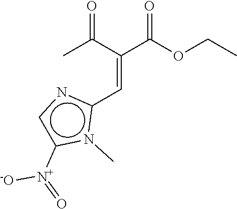 | Propenidazole |
| 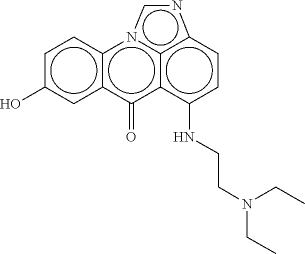 | Imidazoacridinone |
| 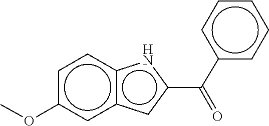 | D-64131 |
| 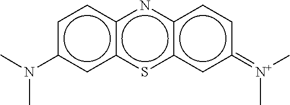 | methylthioninium chloride |
| 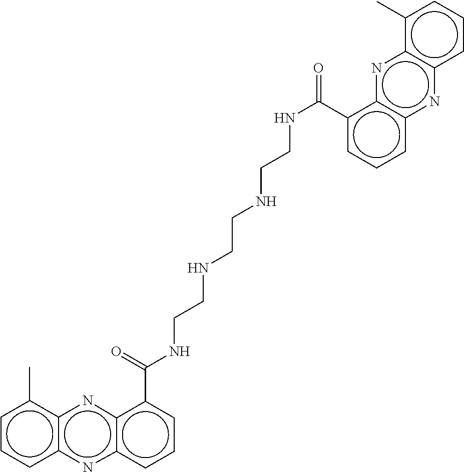 | XR 5944 |
| 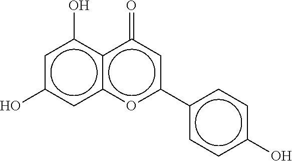 | APIGENIN |
| 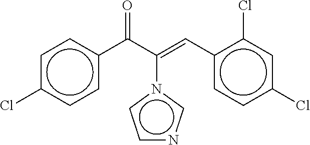 | TS-80 |
| 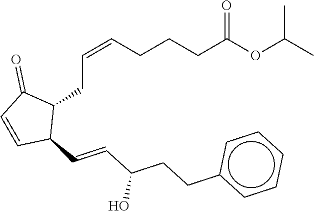 | Posaraprost |
| 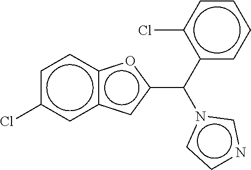 | BECLICONAZOLE |
| 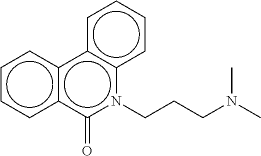 | FANTRIDONE |
| 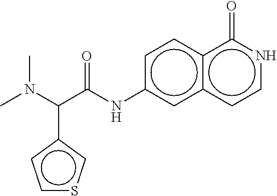 | Verosudil |
| 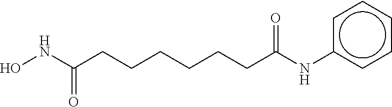 | vorinostat |
| 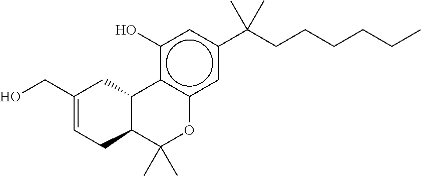 | Dexanabinol |
| 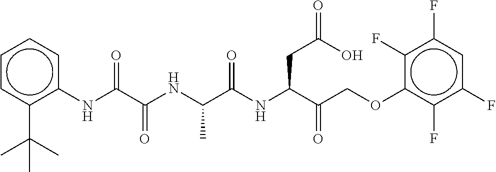 | emricasan |
| 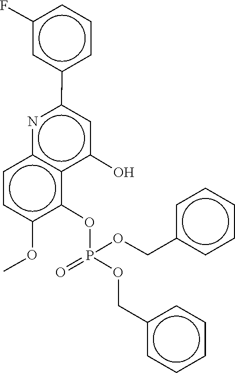 | TRX-818 |
| 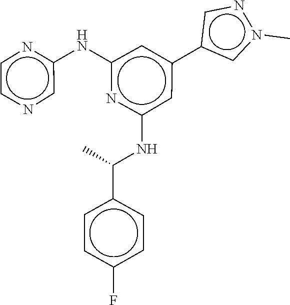 | NS-018 |
| 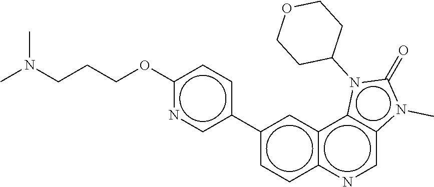 | AZD-0156 |
| 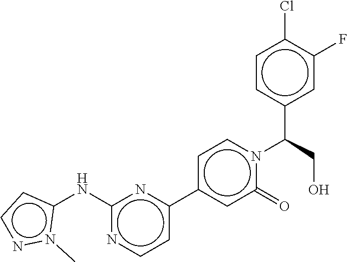 | RG-7842 |
| 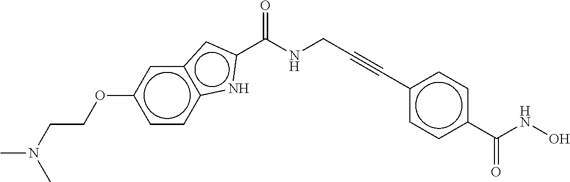 | CRA-026440 |
| 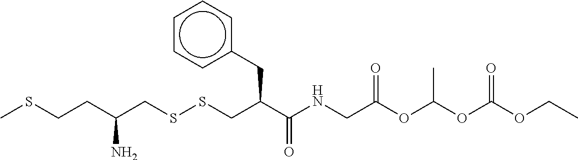 | PL-37 |
| 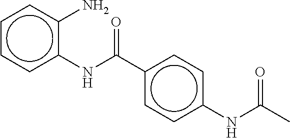 | Tacedinaline |
| 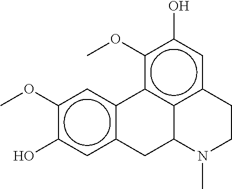 | boldine |
| 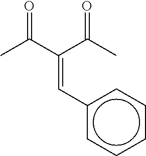 | |
| 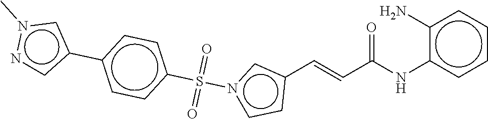 | 4SC-202 |
| 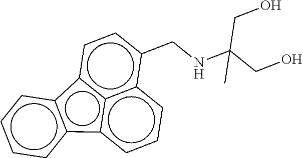 | 773U82 |
| 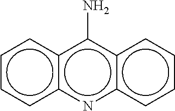 | ACRISORCIN |
| 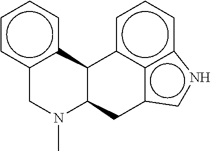 | CY-208-243 |
| 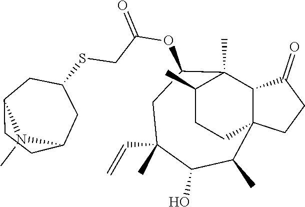 [SMILES: C=C[C@]1(C)C[C@@H](OC(=O)CS[C@@H]2C[C@H]3CC[C@@H](C2)N3C)[C@]2(C)[C@H](C)CC[C@]3(CCC(=O)[C@H]32)[C@@H](C)[C@@H]1O] | retapamulin |
| 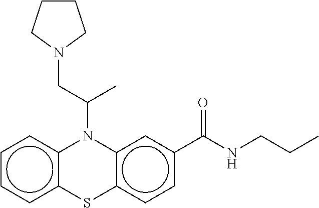 | Apadoline |
| 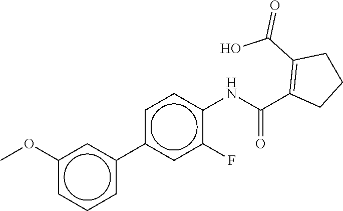 | vidofludimus |
| 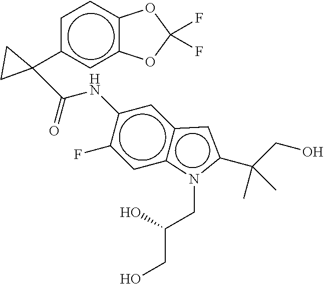 | VX-661 |
| 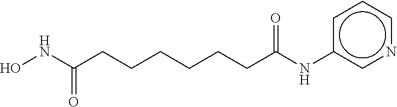 | pyroxamide |
| 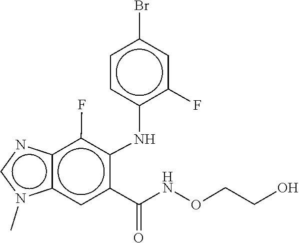 | binimetinib |
| 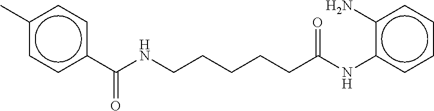 | RG-2833 |
| 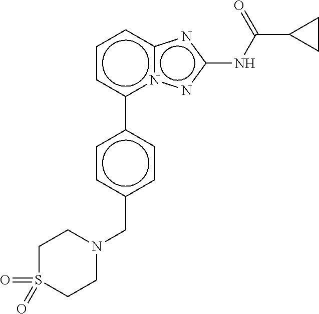 | Filgotinib |
| 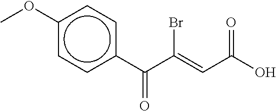 | Bromebrate sodium |
| 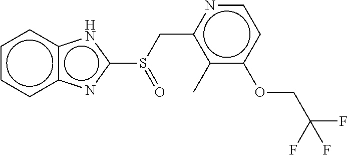 | dexlansoprazole |
| 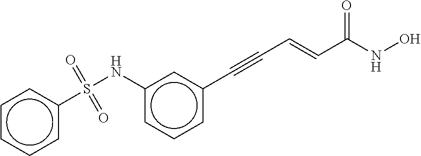 | OXAMFLATIN |
| 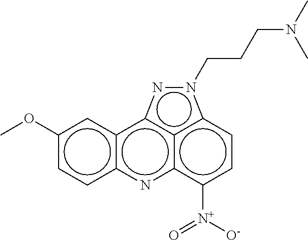 | Pyrazoloacridine |
| 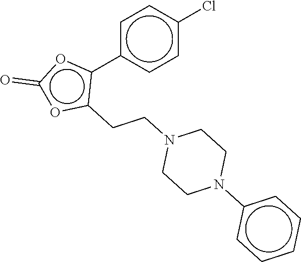 | CLODOXOPONE |
| 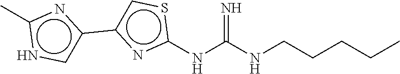 | CP 66948 |
| 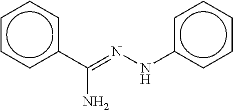 | CBS-1114 |
| 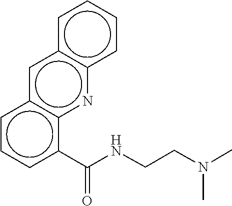 | SN-22995 |
| 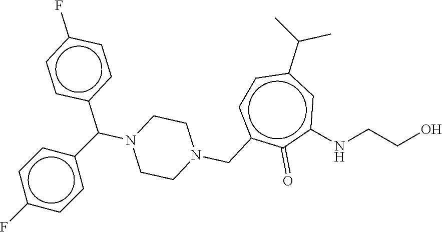 | U 92032 |
| 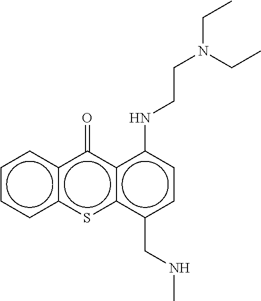 | WIN 33156 |
| 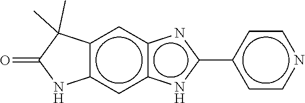 | ADIBENDAN |
| 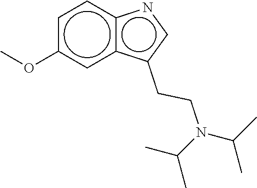 | 5-MeO-DIPT |
| 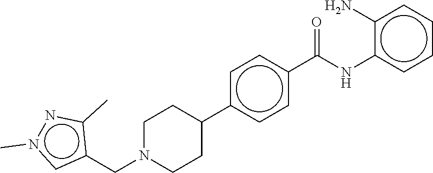 | AZD-9468 |
| 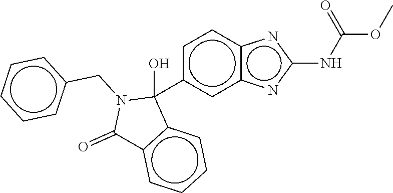 | BMS-908662 |

23. The method according to any of claims 1 to 22, wherein the compound is AR-42: [chemical structure]
